(19) United States
(12) Patent Application Publication
(10) Pub. No.: US 2003/0124195 A1
(43) Pub. Date: Jul. 3, 2003

(54) Hydrocolloid powder composition exhibiting improved dispersibility in an aqueous medium and method for preparing same

(76) Inventors: Francois Delprato, St-Ouen L'Aumone (FR); Karl Heinrich Oskar Tiefenthaler, Kreuzlingen (CH)

Correspondence Address: Kevin McVeigh, Rhodia Inc, Intellectual Property Department, 259 Prospects Plains Road CN 7500, Cranbury, NJ, 08512-7500, US

(21) Appl. No.: 10/276,565

(22) PCT Filed: May 11, 2001
(86) PCT No.: PCT/FR01/01435

(30) Foreign Application Priority Data
May 15, 2000 (FR) 0006173

(51) Int. Cl. A61K 9/14; A61K 9/16; A61K 9/50; B29B 9/00

(52) U.S. Cl. 424/493; 264/5

(57) ABSTRACT

The invention concerns a method for preparing a hydrocolloid powder composition exhibiting good dispersibility in an aqueous medium, without formation of lumps, and whereof the hydration time is controllable, comprising steps which consist in: preparing a potentially reactive crosslinking powder by contacting a highly absorbent support powder with a crosslinking agent capable of crosslinking a hydrocolloid powder, optionally under effect of heat; mixing, optionally by dry process, said powder with a hydrocolloid powder; optionally treating said mixture of powders with water optionally containing salts; and optionally heat-treating said mixture of powders. The invention also concerns said hydrocolloid powder composition comprising at least a crosslinked hydrocolloid constituent, a residual amount of crosslinking agent less than 1000 ppm, a support powder and optionally water.

The present invention relates to pulverulent hydrocolloid compositions exhibiting effective dispersibility in an aqueous medium (no lumps are formed) and a controllable hydration time. It relates more specifically to compositions based on polysaccharides such as cellulose derivatives, starch derivatives, galactomannans, biopolymers such as xanthan gum, etc., which can be dispersed easily and very rapidly in an aqueous medium, which exhibit only a small hydration delay, and which are stable on storage.

The invention likewise relates to a process for preparing such pulverulent hydrocolloid compositions.

It further-relates to an intermediate product, namely a potentially reactive crosslinking powder intended for preparing the aforementioned compositions.

Water-soluble hydrocolloids such as xanthan gum or guar gum, for example, exhibit interesting texturing properties and so find-applications in particular as thickeners or viscosifying agents or as stabilizers, emulsifiers, gelling agents or binders in the-cosmetics, food, agrochemical, and pharmaceutical industries and in the fields of dyeing, detergents, paper, construction materials, drilling fluids, etc.

The incorporation of these hydrocolloids in pulverulent form into aqueous solutions is not easy, however, and poses application problems linked in particular to the formation of lumps. Even under severe stirring conditions with prolonged mixing times, it is sometimes difficult if not impossible to obtain near-complete dispersion of the hydrocolloid powder without the formation of lumps. The formation of lumps makes it, very difficult to hydrate the hydrocolloid powder.

Attempts have therefore been made to enhance the dispersibility of the hydrocolloids.

One of the most commonly employed chemical methods involves using aldehyde-type crosslinking agents, and more particularly dialdehydes, among which glyoxal is the most frequently employed on account of its specific chemical properties.

In the case of xanthan gum, for example, the crosslinking agent, most frequently glyoxal, is introduced during the production of the gum by fermentation of Xanthomonas campestris bacteria. Thus a glyoxal solution is added to the fermentation broth at a pH of approximately 7 or 8. The glyoxal may also be added at any time after the fermenting operation has been completed; for example, following precipitation and drying of the gum, which is then mixed and ground with the glyoxal solution.

In certain cases, the mixture resulting from the addition of glyoxal to the fermentation broth is heated at a temperature which is generally between 70° C. and 100° C.

Although it is possible to enhance the dispersibility of hydrocolloids in an aqueous medium by treating them with aldehydes, problems may arise, depending on the applications, owing to the relatively large residual amount of crosslinking agent which is present in the end product. Specifically, the glyoxal contents are generally between 2 000 and 50 000 ppm in the products obtained by the prior art processes.

Another problem which may present itself is the distribution of the glyoxal relative to the hydrocolloid. Without effective distribution, when small amounts of glyoxal are introduced (less than 2 000 ppm), the resulting powders give rise to the phenomenon of lumps during their dispersion in an aqueous medium as set out above.

Moreover, these powders lack effective stability on storage in the sense that their hydration properties are altered in the course of storage for prolonged periods.

One object of the present invention is to provide hydrocolloid powders which can be dispersed very readily in an aqueous medium without forming lumps.

Another object of the invention is to provide hydrocolloid powders of this type which ultimately contain only a very small residual amount of crosslinking agent.

A further object of the present invention is to provide powders of this type which are stable on storage even in the long term.

Yet another object of the invention is to provide hydrocolloid powders for which the hydration time in an aqueous medium is controllable.

Likewise an object of the invention is to provide a simple and inexpensive process for preparing the above powders.

These aims, and others which will appear on reading the following description, are achieved by the present invention, which provides, in a first embodiment, a process for preparing a pulverulent hydrocolloid composition which is readily dispersible in an aqueous medium, without forming lumps, and whose hydration time is controllable, characterized in that it comprises the steps consisting in:

preparing a potentially reactive crosslinking powder by contacting a highly absorptive support powder with a crosslinking agent capable of crosslinking a hydrocolloid powder under the effect of heat,

mixing said potentially reactive crosslinking powder with a hydrocolloid powder,

optionally treating said powder mixture with water optionally comprising salts,

thermally treating said powder mixture.

In another embodiment, the present invention provides a process for preparing a pulverulent hydrocolloid composition which is readily dispersible in an aqueous medium, without forming lumps, and whose hydration time is controllable, characterized in that it comprises the steps consisting in:

preparing a potentially reactive crosslinking powder by contacting a highly absorptive support powder with a crosslinking agent capable of crosslinking a hydrocolloid powder,

mixing said potentially reactive crosslinking powder with a hydrocolloid powder composed of galactomannans,

optionally treating said powder mixture with water optionally comprising salts.

The invention likewise provides a pulverulent hydrocolloid composition which is readily dispersible in an aqueous medium,.without forming lumps, and whose hydration time is controllable, characterized in that it comprises at least one crosslinked hydrocolloid component, a residual crosslinking agent content of less than 1 000 ppm, a support powder, and, optionally, water.

The invention will be described in greater detail below:

The process of the invention consists first of all in preparing a potentially reactive crosslinking powder by contacting a crosslinking agent with a support powder, said crosslinking powder being intended for reaction, preferably by a dry method, with a hydrocolloid powder.

The support powder is advantageously a fine powder which has a high absorption capacity and is inert, i.e., exhibits no reactive character, either toward the crosslinking agent for which it serves as support or toward the hydrocolloid(s) with which it will subsequently be contacted.

Among powders suitable for the purposes of the invention, nonlimitative mention may be made of silicas, aluminosilicates, kaolin, bentonite, clays, celite, zeolites, alumina, and precipitated calcium carbonate.

The characteristics of the support powder are selected so as to provide a high absorbing power and a fine particle size.

In accordance with the invention it is preferred to use silica of Tixosil® (Rhodia) type, for example, or an aluminosilicate of Tixolex® type, for example.

The invention may typically be implemented using a silica having a particle size of between 10 and 15 microns (measurement by laser granulometry) and an absorption capacity of between 250 and 350 ml of oil per 100 g of silicone (D.O.P: oil uptake method ISO 787/5 using dioctyl phthalate).

The crosslinking agent for its part is selected so as to be able to become fixed on and/or in the support powder and to crosslink, under the action of heat where appropriate, the hydrocolloid(s) with which it will subsequently be contacted.

The crosslinking agent may comprise, for example, one or more aldehydes, which may be selected from monoaldehydes, dialdehydes or polyaldehydes of low molecular weight.

For the purposes of the invention preference is given to dialdehydes, among which glyoxal is selected preferentially.

The amount of crosslinking agent absorbed on and/or in the support powder is adjusted so as to give effective final dispersion of the hydrocolloid composition, in particular as a function of the hydrocolloid powder to be treated and of the characteristics of the support powder.

In accordance with the invention the amount of crosslinking agent provided is generally less than 2 000 ppm by weight relative to the hydrocolloid and preferably between 200 and 1 600 ppm by weight.

According to the invention there is no particular restriction on the way in which the crosslinking agent is contacted with the support powder. Contacting may be effected in particular by impregnation or by spraying.

In accordance with the invention it is preferred to operate by spraying an aqueous solution of crosslinking agent onto the support powder.

In the case of impregnation, a predetermined volume of aqueous solution of crosslinking agent is generally added dropwise to the support powder.

Still in the case of impregnation, it is possible where appropriate to consider soaking the support powder in a predetermined volume of aqueous solution of crosslinking agent.

Once the solution of crosslinking agent has been added to the support powder, the product is mixed for a time sufficient for a homogeneous pulverulent mixture to be obtained. The potentially reactive crosslinking powder obtained at the end must advantageously retain a finely divided appearance close to that of the original support powder. It is possible alternatively to mix the crosslinking agent and the support powder by hand or to operate by any known means, in particular in a conventional mixer, for example, a Lödige mixer.

Contacting the support powder with the crosslinking agent is optionally performed in the presence of an acid, preferably a mineral acid, which promotes the absorption of the crosslinking agent onto and/or into said support powder. This acid is advantageously selected from phosphoric acid, sulfuric acid, and hydrochloric acid or mixtures thereof.

It is also possible where appropriate to add water to produce the crosslinking agent/support powder mixture.

In one preferred embodiment of the invention, from 5 to 50 parts of a 40% strength glyoxal solution, from 0 to 200 parts of water and from 0 to 100 parts of mineral acid are provided per 100 parts of support powder.

The invention provides an intermediate product, namely the potentially reactive crosslinking powder described above, which is intended for the preparation of pulverulent hydrocolloid compositions such as those described in more detail below.

It additionally provides for the use of the aforementioned potentially reactive crosslinking powder for preparing pulverulent hydrocolloid compositions which are readily dispersible in an aqueous medium, without forming lumps, and whose hydration time is controllable, as set out in detail below.

Without wishing to be tied to any one theory, it is thought that the use of a support powder for the crosslinking agent allows better subsequent distribution and dispersal of the crosslinking agent relative to the hydrocolloid, especially when the amount of crosslinking agent is small, as is the case in accordance with the present invention.

The process according to the invention consists, in a second step, in contacting the potentially reactive crosslinking powder with a hydrocolloid powder.

This contacting is preferably conducted by dry mixing.

The hydrocolloid powder may be composed of one hydrocolloid or of a mixture of hydrocolloids, of animal or plant origin.

The hydrocolloid may be selected more particularly from galactomannans such as guar gum, carob gum, tara gum, cellulose derivatives, starch and its derivatives, biopolymers, especially xanthan gum, and succinoglycans and derivatives thereof.

The heteropolysaccharide BM 07 produced by the strain Agrobacterium tumefaciens I-736 (publicly available in accordance with the Treaty of Budapest from the Collection Nationale de Culture des Micro-organismes under this number as of Mar. 1, 1988), which is also known under the name Rheozan® and is described in the European patent application published Jan. 17, 1990 under the number 0351303, is also suitable as a hydrocolloid.

It is to be noted that the heteropolysaccharide produced by the strain

Agrobacterium radiobacter I-2001 (publicly available in accordance with the Treaty of Budapest from the Collection Nationale de Culture des Micro-organismes under this number as of Apr. 3, 1998), and described in the international patent application published Apr. 20, 2000 under the number WO 00/22154, is also suitable as a hydrocolloid.

The heteropolysaccharide produced by the strain Pseudomonas sp I-2054 (publicly available in accordance with the Treaty of Budapest from the Collection Nationale de Culture des Micro-organismes under this number as of Jul. 22, 1998), and described in the international patent application published Oct. 10, 2000 under the number WO 00/63412, is also suitable as a hydrocolloid.

The process according to the invention is preferably applied to xanthan gum or to a xanthan/guar gum mixture.

The powders may be mixed by any known means. It is thus possible to use a conventional mixer, for example, of the Drais type.

Following this step it is possible where appropriate to add—preferably by spraying—water alone or water comprising salts, especially sulfates and/or chlorides such as Na₂SO₄ and NaCl, in order to allow the hydration time of the hydrocolloid powder to be controlled when it is dispersed in an aqueous medium, this final dispersion then no longer being instantaneous in accordance with the definition specified below.

The powder mixture is subsequently treated thermally at a temperature of between 30° C. and 100° C., preferably between 50° C. and 80° C.

It should be noted that, when the hydrocolloid is selected from galactomannans such as guar gum, the thermal treatment is optional.

In other words, the step of mixing crosslinking powder with the galactomannans may be conducted without thermal treatment in the sense indicated earlier on above, for example at ambient temperature (for example, from 18° C. to 25° C.).

The duration of the treatment is determined so as to allow at least partial crosslinking of the hydrocolloid(s) by the crosslinking agent. Generally speaking, a treatment period of not more than 5 hours, advantageously between 5 minutes and 5 hours, preferably between 10 minutes and 1 hour, is provided.

The process according to the invention has the advantage of not necessitating any supplementary operation of grinding or of drying, so making it a process which is simpler and less costly than the processes proposed to date.

The pulverulent hydrocolloid composition of the invention comprises a crosslinked hydrocolloid component a residual crosslinking agent content of generally less than 1 000 ppm by weight, approximately relative to the hydrocolloid, the support powder, and, where appropriate, water.

The crosslinking agent content may be determined by liquid-chromatographic assay of 2,4-dinitrophenylhydrazone following extraction with methanol.

The expression “hydrocolloid component” here denotes a single hydrocolloid compound or a mixture of hydrocolloid compounds, as indicated above in the context of the process of the invention.

The residual crosslinking agent content represents the proportion of crosslinking agent which has not reacted with the hydrocolloid or hydrocolloids.

This residual content commonly does not exceed 1 000 ppm, which is very low in particular in relation to the existing processes. This content may be more particularly not more than 700 ppm and more particularly still not more than 500 ppm.

The support powder which is present in the final composition corresponds to the powder which served as support for the crosslinking agent, as defined above in the context of the potentially reactive crosslinking powder. It represents not more than 20% by weight of the final composition, preferably from 0.5% to 5% by weight.

The final composition may likewise comprise water originating essentially from the constituents which have been reacted. It generally represents from 5 to 20% by weight of the final composition, preferentially from 8 to 15% by weight.

The pulverulent hydrocolloid composition of the invention possesses the property of being able to be dispersed instantaneously in an aqueous medium.

In accordance with the invention, “effective dispersion” means dispersion without lumps being formed, which can-be obtained by simple mixing with said aqueous medium, by hand for example.

The hydration time for its part is from several seconds to a number of minutes.

In the case of rapid hydration, the hydration time is from several seconds, 1 second for example, to not more than 3 minutes, more preferentially from around ten seconds, 10 seconds for example, to not more than 2 and a half minutes.

In the case of slow hydration, the hydration time is from at least 3 minutes to not more than 40 minutes, more preferentially not more than 20 minutes.

This contrasts with the hydrocolloid powders available to date, which have required drastic dispersing conditions and/or prolonged hydration times or have given rise to the appearance of agglomerates which do not allow homogeneous dispersion. The hydration test consists in measuring the time required for the vortex to disappear when 1.5 g of the test powder are added to 150 ml of synthetic water (whose composition is 323.4 mg of CaCl₂.2H₂O in 1 l of distilled water) with magnetic stirring at 750 revolutions/minute. By way of example, Rhodopol® 50 MD (sold by the company Rhodia) has a hydration time of 20 minutes.

The expression “aqueous medium” used herein denotes water alone or water to which salt has been added (brine), whose pH is generally close to neutral.

As has been described above, the hydrocolloid compositions of the invention may exhibit controlled dispersion, i.e., dispersion which is not “instantaneous” in the sense indicated above but which is nevertheless of determined duration.

The pulverulent hydrocolloid compositions of the invention have the advantage of being stable on storage; that is, they retain their properties, especially those set out above, for a period of at least 6 months.

The compositions of the invention find application as thickeners or viscosifying agents or as stabilizers, emulsifiers, gelling agents or binders in the cosmetics, food, agrochemical, and pharmaceutical industries and in the fields of dyeing, detergents, paper construction materials, drilling fluids, etc.

The present invention is illustrated below by means of the following examples, which should not be considered as being limitative.

### EXAMPLES

### Example 1

2.3 parts of a mixture composed of 100 parts of Tixosil® silica (sold by Rhodia), 100 parts of water and 30 parts of a solution containing 40% of glyoxal by mass are mixed homogeneously with 100 parts of xanthan gum. The mixture is heated at 60° C. for 60 minutes and then sieved to remove particles larger than 450 μm. The sieve residue is usually less than 0.5%. The product obtained undergoes dispersion in water without lumps. The residual glyoxal content of the end product is 464 ppm. The hydration time is 20 seconds.

### Example 2

2.3 parts of a mixture composed of 100 parts of Tixosil® silica (sold by Rhodia), 87.5 parts of water, 12.5 parts of 75% strength phosphoric acid and 30 parts of a solution containing 40% of glyoxal by mass are mixed homogeneously with 100 parts of guar gum. The mixture is heated at 70° C. for 1 hour and then sieved to remove particles larger than 450 μm. The sieve residue is usually less than 0.5%. The product obtained undergoes dispersion in water without lumps. The hydration time is 2 minutes and 10 seconds.

### Example 3

1.8 parts of a mixture composed of 100 parts of Tixolex®-17 aluminosilicate (sold by Rhodia), 50 parts of water and 30 parts of a solution containing 40% of glyoxal by mass are mixed homogeneously with 100 parts of xanthan gum. The mixture is heated at 70° C. for 1 hour and then sieved to remove particles larger than 450 μm. The sieve residue is usually less than 0.5%. The product obtained undergoes dispersion in water without lumps. The residual glyoxal content of the end product is 860 ppm. The hydration time is 25 seconds.

### Example 4

2.3 parts of a mixture composed of 100 parts of Tixosil® silica (sold by Rhodia), 100 parts of water and 30 parts of a solution containing 40% of glyoxal by mass are mixed homogeneously with 100 parts of a mixture containing 90% of xanthan gum and 10% of guar gum. The mixture is heated at 60° C. for 30 minutes and then sieved to remove particles larger than 450 μm. The sieve residue is usually less than 0.5%. The product obtained undergoes dispersion in water without lumps. The residual glyoxal content of the end product is 685 ppm. The hydration time is 35 seconds.

### Example 5

4 parts of a mixture composed of 100 parts of Tixosil® silica (sold by Rhodia), 55 parts of water, 50 parts of a 75% by mass phosphoric acid solution and 10.75 parts of a solution containing 40% of glyoxal by mass are mixed homogeneously with 100 parts of guar gum. The mixture is left at ambient temperature for at least 5 days and then sieved to remove particles larger than 450 μm. The sieve residue is usually less than 1%. The product obtained undergoes dispersion without lumps. The hydration time is approximately 28 minutes.

### Example 6

4 parts of a mixture composed of 100 parts of Tixosil® silica (sold by Rhodia), 100 parts of water, 50 parts of a 75% by mass phosphoric acid solution and 16.6 parts of a solution containing 40% of glyoxal by mass are mixed homogeneously with 100 parts of xanthan gum. The mixture is heated to 80° C. for 1 hour and then sieved to remove particles larger than 450 μm. The sieve residue is usually less than 1%. The product obtained undergoes dispersion without lumps. The hydration time is approximately 8 minutes.

### Example 7

4 parts of a mixture composed of 100 parts of Tixosil® silica (sold by Rhodia), 106.4 parts of water, 42.5 parts of a 75% by mass phosphoric acid solution and 20 parts of a solution containing 40% of glyoxal by mass are mixed homogeneously with 100 parts of Rheozan® (sold by Rhodia). The mixture is heated to 70° C. for 2 hours and then sieved to remove particles larger than 450 μm. The sieve residue is usually less than 0.5%. The product obtained undergoes dispersion without lumps. The hydration time is approximately 45 seconds. The residual glyoxal content of the end product is 490 ppm.

1. A process for preparing a pulverulent hydrocolloid composition which is readily dispersible in an aqueous medium, without forming lumps, and whose hydration time is controllable, characterized in that it comprises the steps consisting in:
preparing a potentially reactive crosslinking powder by contacting a highly absorptive support powder with a crosslinking agent capable of crosslinking a hydrocolloid powder under the effect of heat,
mixing said potentially reactive crosslinking powder with a hydrocolloid powder,
optionally treating said powder mixture with water optionally comprising salts,
thermally treating said powder mixture.

2. A process for preparing a pulverulent hydrocolloid composition which is readily dispersible in an aqueous medium, without forming lumps, and whose hydration time is controllable, characterized in that it comprises the steps consisting in:
preparing a potentially reactive crosslinking powder by contacting a highly absorptive support powder with a crosslinking agent capable of crosslinking a hydrocolloid powder,
mixing said potentially reactive crosslinking powder with a hydrocolloid powder composed of galactomannans,
optionally treating said powder mixture with water optionally comprising salts.

3. The process as claimed in claim 1 or 2, characterized in that the support powder is selected from silicas, aluminosilicates, kaolin, bentonite, clays, celite, zeolites, alumina, and precipitated calcium carbonate.

4. The process as claimed in any one of the preceding claims, characterized in that the support powder comprises a silica or an aluminosilicate.

5. The process as claimed in any one of the preceding claims, characterized in that the support powder comprises silica having a particle size of between 10 and 15 microns (measurement by laser granulometry) and an adsorption capacity of between 250 and 350 ml of oil per g of silica (D.O.P: oil uptake method ISO 787/5 using dioctyl phthalate).

6. The process as claimed in any one of the preceding claims, characterized in that the crosslinking agent comprises one or more aldehydes selected from monoaldehydes, dialdehydes, and polyaldehydes of low molecular weight, the dialdehydes being preferred.

7. The process as claimed in any one of the preceding claims, characterized in that the crosslinking agent is glyoxal.

8. The process as claimed in any one of the preceding claims, characterized in that the crosslinking agent is in aqueous solution and is contacted with the support powder by spraying.

9. The process as claimed in any one of claims 1 to 7, characterized in that the crosslinking agent is contacted with the support powder by impregnation.

10. The process as claimed in any one of the preceding claims, characterized in that the crosslinking agent is contacted with the support powder in the presence of an acid, preferably a mineral acid, and/or water.

11. The process as claimed in any one of the preceding claims, characterized in that a mineral acid is used selected from phosphoric acid, sulfuric acid, and hydrochloric acid or mixtures thereof.

12. The process as claimed in any one of the preceding claims, characterized in that from 5 to 50 parts of a 40% strength glyoxal solution, from 0 to 200 parts of water and from 0 to 100 parts of mineral acid are provided per 100 parts of support powder.

13. The process as claimed in claim 1 and any one of claims 3 to 12, characterized in that the hydrocolloid powder comprises one or more hydrocolloids selected from galactomannans such as guar gum, carob gum, tara gum, cellulose derivatives, starch and its derivatives, biopolymers, especially xanthan gum, and also succinoglycans and derivatives thereof.

14. The process as claimed in claim 1 and any one of claims 3 to 13, characterized in that the hydrocolloid powder is a xanthan gum powder.

15. The process as claimed in claim 1 and any one of claims 3 to 13, characterized in that the hydrocolloid powder is a powder comprising a mixture of xanthan and guar gums.

16. The process as claimed in any one of the preceding claims, characterized in that the amount of crosslinking agent is less than 2 000 ppm by weight, relative to the hydrocolloid powder treated and is preferably between 200 and 1 600 ppm by weight.

17. The process as claimed in claim 1 and any one of claims 3 to 16, characterized in that the mixture of the potentially reactive crosslinking powder and the hydrocolloid powder is heated at a temperature of between 30° C. and 100° C., preferably between 50° C. and 80° C.

18. The process as claimed in claim 1 and any one of claims 3 to 17, characterized in that the mixture of the potentially reactive crosslinking powder and the hydrocolloid powder is treated thermally for a duration of not more than 5 hours, preferably of between 5 minutes and 5 hours, more preferably of between 10 minutes and 1 hour.

19. A potentially reactive crosslinking powder intended for the preparation of a pulverulent hydrocolloid composition which is readily dispersible in an aqueous medium, without forming lumps, and whose hydration time is controllable, comprising a highly absorptive support powder selected from silicas, aluminosilicates, kaolin, bentonite, clays, celite, zeolites, alumina, and precipitated calcium carbonate and a crosslinking agent comprising one or more aldehydes selected from monoaldehydes, dialdehydes, and polyaldehydes of low molecular weight, the dialdehydes being preferred, as an intermediate.

20. A pulverulent hydrocolloid composition which is readily dispersible in an aqueous medium, without forming lumps, and whose hydration time is controllable, characterized in that it comprises at least one crosslinked hydrocolloid component, a residual crosslinking agent content of less than 1 000 ppm, a support powder and, where appropriate, water.

21. The composition as claimed in claim 20, characterized in that the hydrocolloid component comprises one or more hydrocolloids selected from galactomannans such as Guar gum, carob gum, tara gum, cellulose derivatives, starch and its derivatives, biopolymers, especially xanthan gum, and also succinoglycans and derivatives thereof.

22. The composition as claimed in one of claims 20 or 21, characterized in that the hydrocolloid component is composed of xanthan gum.

23. The composition as claimed in one of claims 20 or 21, characterized in that the hydrocolloid component is composed of a mixture of xanthan and guar gums.

24. The composition as claimed in any one of claims 20 to 23, characterized in that the crosslinking agent comprises one or more aldehydes selected from monoaldehydes, dialdehydes, and polyaldehydes of low molecular weight, the dialdehydes being preferred.

25. The composition as claimed in any one of claims 20 to 24, characterized in that the crosslinking agent is glyoxal.

26. The composition as claimed in any one of claims 20 to 25, characterized in that the proportion of support powder represents not more than 20% by weight of the pulverulent composition and is preferably between 0.5% and 5% by weight.

27. The composition as claimed in any one of claims 20 to 26, characterized in that the support powder is selected from silicas, aluminosilicates, kaolin, bentonite, clays, celite, zeolites, alumina, and precipitated calcium carbonate.

28. The composition as claimed in any one of claims 20 to 27, characterized in that the support powder comprises a silica or an aluminosilicate.

29. The composition as claimed in any one of claims 20 to 28, characterized in that it contains between 5 and 20% by weight of water relative to the pulverulent composition, preferably between 8 and 15% by weight.

30. The composition as claimed in any one of claims 20 to 29, characterized in that it is stable on storage for at least 6 months.

31. Composition as claimed in any one of claims 20 to 30, characterized in that it has a rapid hydration time varying from not more than 3 minutes, preferably from around ten seconds to not more than 2 and a half minutes.

32. The composition as claimed in any one of claims 20 to 30, characterized in that it has a slow hydration time varying from not more than 40 minutes, preferably not more than 20 minutes.

33. The use of a potentially reactive crosslinking powder comprising a highly absorptive support powder selected from silicas, aluminosilicates, kaolin, bentonite, clays, elite, zeolites, alumina and precipitated calcium carbonate and a crosslinking agent comprising one or more aldehydes selected from monoaldehydes, dialdehydes, and polyaldehydes of low molecular weight for preparing a pulverulent hydrocolloid composition which is readily dispersible in an aqueous medium, without forming lumps, and whose hydration time is controllable.

34. The use of a pulverulent hydrocolloid composition as claimed in any one of claims 20 to 32 as thickeners or viscosifying agents, stabilizers, emulsifiers, gelling agents or binders in the cosmetics, food, agrochemical, and pharmaceutical industries and in the fields of dyeing, detergents, paper, construction materials, and drilling fluids.

35. The use of a potentially reactive crosslinking powder as claimed in claim 19 for preparing a hydrocolloid powder as claimed in any one of claims 20 to 32, intended for use as thickeners or viscosifying agents, stabilizers, emulsifiers, gelling agents or binders in the cosmetics, food, agrochemical, and pharmaceutical industries and in the fields of dyeing, detergents, paper, construction materials, and drilling fluids.
